(19) United States
(12) Patent Application Publication
(10) Pub. No.: US 2018/0008526 A1
(43) Pub. Date: Jan. 11, 2018

(54) COMPOSITIONS COMPRISING AT LEAST ONE OIL AND AT LEAST ONE POLYESTER

(71) Applicant: L'OREAL, Paris (FR)

(72) Inventors: Sophie BEAUMARD, Villejuif (FR); Claudia BARBA, Paris (FR)

(21) Appl. No.: 15/541,891

(22) PCT Filed: Jan. 7, 2016
(86) PCT No.: PCT/EP2016/050237

§ 371 Date: Jul. 6, 2017

(30) Foreign Application Priority Data
Jan. 7, 2015 (FR) 1550124

(51) Int. Cl. A61K 8/85; A61K 8/31; A61K 8/92; A61Q 1/10

(52) U.S. Cl. CPC A61K 8/85; A61Q 1/10; A61K 8/31; A61K 8/922; A61K 8/927

(57) ABSTRACT

This invention relates to a composition, in particular for coating keratin fibers, in particular eyelashes, comprising:
at least one oil,
at least one wax, and
at least one liquid polyester obtained by condensing unsaturated fatty acid dimers and/or trimers and diol, according to a content strictly less than 12% by weight with respect to the total weight of said composition.

This invention concerns compositions, in particular cosmetic, comprising at least one oil and at least one polyester.

This invention also concerns the use of these compositions for the coating of keratin fibers, and in particular of eyelashes or eyebrows.

In particular, the compositions according to the invention are makeup compositions, and possibly eyelash care. This invention also relates to a method of coating keratin fibers, in particular a makeup method, and possibly eyelash care.

The cosmetic compositions intended for makeup of the eyelashes such as mascaras are in particular prepared according to types of formulation: on the one hand, aqueous mascaras referred to as “cream mascaras”, in the form of dispersion of waxes in the water; and, on the other hand, anhydrous mascaras or with low water content, referred to as “waterproof mascaras”, in the form of dispersions of waxes in organic solvents.

The mascara application aims in particular to increase the volume of the eyelashes and consequently to increase the intensity of the look. For this, there are many thickening or volumizing mascaras of which the principle consists in depositing the maximum of material on the eyelashes in such a way as to obtain this volumizing (or loading) effect.

However, waterproof mascaras have the disadvantage of not providing as much volume as aqueous mascaras.

To date, in order to increase the volumizing result of waterproof mascaras, waxes, fillers or pigments are added to the latter in order to increase the dry matter content. However, on the other hand, this high content in dry matter has the disadvantage of reducing the playtime or the stability. Such mascaras then have a texture that quickly becomes dry and difficult to work with after several passages on the eyelashes.

There is therefore a need for a new cosmetic composition of the waterproof mascara type, that has a volumizing effect, without reducing the playtime and the stability.

“Better playtime” means working better with the composition via successive brushes, by reducing and even annihilating the braking effects to application.

This invention therefore has for purpose to supply a composition, in particular for the coating of keratin fibers, of the waterproof mascara type, giving rise to a volume effect on the eyelashes.

This invention also has for purpose to supply a composition, in particular for the coating of keratin fibers, of the waterproof mascara type, having good application properties in terms of playtime.

This invention also has for purpose to supply a composition, in particular for the coating of keratin fibers, of the waterproof mascara type, having good stability properties.

As such, this invention relates to a composition, in particular for coating keratin fibers, in particular eyelashes, comprising:
at least one oil,
at least one wax, and
at least one liquid polyester obtained by condensing unsaturated fatty acid dimers and/or trimers and diol, according to a content strictly less than 12% by weight with respect to the total weight of said composition.

It has therefore been observed that the adding of a polyester such as defined hereinabove makes it possible to improve the volume effect on the eyelashes of the compositions according to the invention of the waterproof mascara type, and this without reducing the makeup and stability properties of these compositions, in particular when they are used for coating keratin fibers.

This invention also has for object a method for coating keratin fibers, and in particular makeup of the eyelashes, comprising a step of applying a cosmetic composition for coating keratin fibers such as described hereinabove.

This invention also has for object the use of a composition such as described hereinabove in order to obtain a composition that gives rise to a volume effect on the eyelashes.

Polyester

The composition according to the invention comprises at least one polyester such as defined hereinabove. They may thus comprise a single polyester or a mixture of a plurality of different polyesters.

Liquid polyester means a polyester that begins to flow under its own weight in less than one minute at ambient temperature (25° C.).

“Unsaturated fatty acids” designates, in the framework of this invention, mono- or polyunsaturated fatty acids comprising 14 to 22 carbon atoms. The unsaturated fatty acid dimers can in particular comprise from 2 to 4 unsaturations in their carbon chain. The unsaturated fatty acid trimers can in particular comprise from 3 to 6 unsaturations in their carbon chain. Preferably, the unsaturated fatty acid dimers and/or trimers are polycarboxylic acids comprising at least 2 and up to 6 carboxylic acid functions per molecule.

In a preferred embodiment, the unsaturated fatty acid dimer can comprise from 28 to 44 carbon atoms and 2 carboxylic acid functions. The unsaturated fatty acid trimer can comprise from 42 to 66 carbon atoms and 3 carboxylic acid functions.

According to a preferred embodiment, the polyester is obtained by condensing an unsaturated fatty acid dimer comprising 36 carbon atoms and 2 carboxylic acid functions, and a diol.

Mixtures of unsaturated fatty acid and/or saturated fatty acid dimers and trimers (non-polymerized therefore corresponding to a monomer) can also be implemented in the framework of the invention. In the case of such a mixture, a mixture is preferred containing more than 50% by weight in dimers, for example a mixture comprising more than 90% by weight, preferably more than 95%, of acids in the form of dimers, with the rest of the mixture able to be unsaturated fatty acid trimers and/or monomers.

The unsaturated fatty acid dimer and/or trimer can possibly be hydrogenated after the polymerization reaction of the unsaturated fatty acid in order in particular to improve the stability of the dimer or trimer product.

Hydrogenated fatty acid dimers (oleic or linoleic acid) are in particular marketed under the brands EMPOL1008, EMPOL1004, EMPOL1025, EMPOL1011 and EMPOL1062 by Cognis and Pripol 1006 (dilinoleic acid) by Uniqema, International. Uniqema also markets a hydrogenated fatty acid dimer under the name Pripol 1013 (hydrogenated dilinoleic acid).

Particularly preferably, unsaturated fatty acid dimer is a linoleic acid dimer, also called dilinoleic acid, obtained by intermolecular polymerizing of the linoleic acid.

The unsaturated fatty acid can be of natural origin, preferably of plant origin. A fatty acid of plant origin can come from any plant source that produces said fatty acid. For example, in the case of linoleic acid, molecules can be used extracted from soybean or from rapeseed.

The polyester in the composition according to the invention is therefore obtained by condensing a long chain fatty acid polymerized with a diol.

In the framework of this invention, “diol” designates a hydrocarbon compound in C2 to C10, preferably in C2-C8, and preferentially in C2-C6, of which the carbon chain is substituted by two hydroxyl functions. The hydrocarbon chain or chains can be interrupted with an oxygen atom.

The diols that can be used according to the invention can be saturated or unsaturated linear, branched or cyclic alcohols. Preferably, the diol is a saturated linear diol.

Particularly preferably, the diol is a butanediol, in particular 1,2-butanediol, 1,3-butanediol or 1,4-butanediol, and preferably 1,4-butanediol.

Advantageously, the polyester implemented in the composition according to the invention has an average molecular weight between 500 and 2,000, preferably between 1,000 and 2,000, and preferentially between 1,200 and 1,800.

In a particularly preferred embodiment, the polyester obtained by condensing unsaturated fatty acid dimer and/or trimer and diol is a polymer, or polyester, of dilinoleic acid and 1,4-butanediol.

According to a preferred embodiment, the polyester according to the invention is a polyester of dilinoleic acid and of 1,4-butanediol that preferable has an average molecular weight of 1,500, a viscosity at 40° C. of 2500-3500 cSt and a refraction index at 25° C. of 1.475-1.485.

Mention may particularly be made in this respect of the polymer sold by Biosynthesis under the name Viscoplast 14436H (INCI name: dilinoleic acid/butanediol copolymer).

According to an embodiment, the content by weight of polyester in the compositions of the invention is strictly less than 12%, preferably less than or equal to 10%, preferably less than or equal to 7%, and preferentially equal to 5% by weight, with respect to the total weight of said composition.

According to a preferred embodiment, the composition according to the invention comprises 3% to 12%, preferentially 5% to 10% by weight of polyester with respect to the total weight of said composition.

Oil

The composition according to the invention comprises at least one oil. “Oil” is means a liquid fatty body at ambient temperature and atmospheric pressure. The composition according to the invention can comprise at least one volatile oil and/or at least one additional non-volatile oil, and mixtures thereof.

Volatile Oil

The composition according to the invention can comprise at least one volatile oil.

The term “volatile oil” denotes an oil (or non-aqueous medium) liable to evaporate on skin contact in less than one hour, at ambient temperature and atmospheric pressure. The volatile oil is a volatile cosmetic oil that is liquid at ambient temperature. More precisely, a volatile oil has an evaporation rate of between 0.01 and 200 mg/cm²/mn, inclusive.

To measure this evaporation rate, 15 g of oil or an oil mixture to be tested are introduced into a crystallizer with a diameter of 7 cm, placed on a scale located in a large chamber of around 0.3 m³, with controlled temperature, at 25° C., and hygrometry, at 50% relative humidity. The liquid is left to evaporate freely, without stirring, by allowing ventilation with a fan (PAPST-MOTOREN, reference 8550 N, rotating at 2700 rpm) arranged vertically above the crystallizer containing said oil or said mixture, with the blades being directed toward the crystallizer and at a distance of 20 cm with respect to the crystallizer base. The mass of oil remaining in the crystallizer is measured at regular intervals. The evaporation rates are expressed in mg of oil evaporated per unit of surface (cm²) and per unit of time (minutes).

This volatile oil can be a hydrocarbon oil.

The hydrocarbon volatile oil may be chosen from hydrocarbon oils having from 7 to 16 carbon atoms.

The composition according to the invention can contain one or several volatile branched alkanes. “One or several volatile branched alkanes” means indifferently “one or several volatile branched alkane oils”.

As a hydrocarbon volatile oil having 7 to 16 carbon atoms, mention can be made in particular of C8-C16 branched alkanes such as C8-C16 iso-alkanes (also called isoparaffins), isododecane, isodecane, isohexadecane and for example the oils sold under the trade names of Isopars or Permetyls, C8-C16 branched esters such as isohexyl neopentanoate, and mixtures thereof. Preferably, the hydrocarbon volatile oil having from 8 to 16 carbon atoms is chosen from among isododecane, isodecane, isohexadecane and mixtures thereof, and is in particular isododecane.

The composition according to the invention can contain one or several volatile linear alkanes. “One or several linear branched alkanes” means indifferently “one or several volatile linear alkane oils”.

A volatile linear alkane suitable for the invention is liquid at ambient temperature (about 25° C.) and at atmospheric pressure (760 mm Hg).

“Volatile linear alkane” suitable for the invention means a cosmetic linear alkane, liable to evaporate on skin contact in less than one hour, at ambient temperature (25° C.) and atmospheric pressure (760 mm Hg, i.e. 101 325 Pa), liquid at ambient temperature, having in particular an evaporation rate ranging from 0.01 to 15 mg/cm²/min, at ambient temperature (25° C.) and atmospheric pressure (760 mm Hg).

The linear alkanes, preferably of plant origin, comprise from 7 to 15 carbon atoms, in particular from 9 to 14 carbon atoms, and more particularly from 11 to 13 carbon atoms.

As an example of linear alkane suitable for the invention, mention can be made of the alkanes described in patent applications of Cognis WO 2007/068371, or WO2008/155059 (separate alkane mixtures and different by at least one carbon). These alkanes are obtained from fatty alcohols, themselves obtained from coconut or palm oil.

As an example of linear alkane suitable for the invention, mention can be made of n-heptane (C7), n-octane (C8), n-nonane (C9), n-decane (C10), n-undecane (C11), n-dodecane (C12), n-tridecane (C13), n-tetradecane (C14), n-pentadecane (C15), and mixtures thereof, and in particular the mixture of n-undecane (C11) and of n-tridecane (C13) described in example 1 of the application WO2008/155059 of Societe Cognis. Mention can also be made of n-dodecane (C12) and n-tetradecane (C14) old by Sasol respectively under references PARAFOL 12-97 and PARAFOL 14-97, as well as mixtures thereof.

The linear alkane can be used alone or in a mixture of at least two separate alkanes and different from each other by a number of carbons of at least 1, and in particular a mixture of at least two linear alkanes comprising from 10 to 14 separate carbon atoms and different from each other by a number of carbons of at least 2, and in particular a mixture of C11/C13 linear volatile alkanes or a mixture of C12/C14 linear alkanes, in particular an n-undecane/n-tridecane mixture (such a mixture can be obtained according to example 1 or example 2 of WO 2008/155059).

Alternatively or additionally, the composition made can comprise at least one volatile silicone solvent or oil, compatible with a cosmetic use.

Silicone oil means an oil that contains at least one silicon atom, and in particular containing Si—O groups. According to one embodiment, said composition comprises less than 10% by weight of non-volatile silicone oils, with respect to the total weight of the composition, preferably less than 5% by weight, or is even free from silicone oil.

A silicon volatile oil, mention can be made of cyclic polysiloxanes, linear polysiloxanes and mixtures thereof. A linear volatile polysiloxanes, mention can be made of hexamethyldisiloxane, octamethyltrisiloxane, decamethyltetrasiloxane, tetradecamethylhexasiloxane and hexadecamethylheptasiloxane. As cyclic volatile polysiloxanes, mention can be made of hexamethylcyclotrisiloxane, octamethylcyclotetrasiloxane, decamathylcyclopentasiloxane and dodemethylcyclohexasiloxane.

Alternatively or additionally, the composition made can comprise at least one fluorinated volatile oil.

The term fluorinated oil refers to an oil containing at least one fluorine atom.

As a volatile fluorinated oil, mention can be made of nonafluoromethoxybutane or perfluoromethylcyclopentane, and mixtures thereof.

The volatile oil(s) can be present with a content ranging from 0.1% to 80% by weight, with respect to the total weight of the composition, preferably ranging from 0.5% to 70% by weight, preferentially ranging from 0.5% to 60% by weight and more preferentially from 10% to 50% by weight with respect to the total weight of the composition. In particular, the volatile oil(s) can be present in the composition with a content greater than or equal to 30% by weight, even greater than or equal to 35% by weight, even greater than or equal to 40% by weight, even greater than or equal to 45% by weight with respect to the total weight of the composition.

According to a preferred alternative, the composition comprises at least isododecane, and preferably with a content by weight greater than or equal to 20%, even greater than or equal to 35% by weight, even greater than or equal to 40% by weight, even greater than or equal to 45% by weight, with respect to the total weight of said composition.

Additional Non-Volatile Oil

In addition to the polyester such as defined hereinabove, the composition according to the invention can comprise at least one non-volatile oil.

The term “non-volatile oil” denotes an oil remaining on the skin or keratin fiber at ambient temperature and pressure. More precisely, a non-volatile oil has an evaporation rate strictly below 0.01 mg/cm²/min.

The additional non-volatile oil suitable for this invention can be chosen from among hydrocarbon oils and silicone oils.

The non-volatile hydrocarbon oils suitable for this invention can in particular be chosen from among:
hydrocarbon oils of plant origin such as triglycerides constituted of fatty acid esters and glycerol for which the fatty acids can have chain lengths ranging from C4 to C28, with the latter able to be linear or branched, saturated or unsaturated; these oils are in particular wheat germ, sunflower, grape seed, sesame, corn, apricot, castor, shea, avocado, olive, soybean oils, sweet almond, palm, rapeseed, cotton, hazelnut, macadamia, jojoba, alfalfa, poppy seed, pumpkin, sesame, squash, rapeseed, blackcurrant, evening primrose, millet, barley, quinoa, rye, safflower, candlenut, passiflora, musk rose oil; or caprylic/capric acid triglycerides such as those sold by Stearineries Dubois or those sold under the trade names Miglyol 810®, 812® and 818® by Sasol;
synthetic ethers having from 10 to 40 carbon atoms;
linear or branched hydrocarbons of mineral or synthetic origin, such as petroleum jelly, polydecenes, hydrogenated polyisobutene such as Parleam, squalane, and mixtures thereof;
synthetic esters such as the oils having the formula R1COOR2 wherein R1 represents the residue of a linear or branched fatty acid comprising 1 to 40 carbon atoms and R2 represents a hydrocarbon chain, particularly branched containing 1 to 40 carbon atoms where R1+R2 10, such as for example Purcellin oil (cetostearyl octanoate), isopropyl myristate, isopropyl palmitate, C12 to C15 alcohol benzoate, hexyl laurate, diisopropyl adipate, isononyl isononanoate, 2-ethylhexyl palmitate, isostearate of isostearate, alcohol or polyalcohol octanoates, decanoates or ricinoleates such as propylene glycol dioctanoate; hydroxylated esters, such as isostearyl lactate, diisostearyl malate; and pentaerythritol esters;
fatty alcohols that are liquid at ambient temperature, with a branched and/or unsaturated carbon chain having 12 to 26 carbon atoms, such as octyldodecanol, isostearyl alcohol, oleic alcohol, 2-hexyldecanol, 2-butyloctanol, or 2-undecylpentadecanol;
higher fatty acids, such as oleic acid, linoleic acid, linolenic acid, and mixtures thereof.

The non-volatile silicone oils suitable for this invention can in particular be chosen from among:
non-volatile silicone oils that can be used in the composition in accordance with the invention can be polydimethylsiloxanes (PDMS) which are non-volatile, polydimethylsiloxanes comprising alkyl or alkoxy groups which are pendant or at the end of the silicone chain, said groups each having 2 to 24 carbon atoms; phenylated silicones, such as phenyl trimethicones, phenyl dimethicones, phenyltrimethylsiloxydiphenylsiloxanes, diphenyl dimethicones, diphenylmethyldiphenyl-trisiloxanes or (2-phenylethyl)trimethylsiloxysilicates.

The content in non-volatile oil in the composition according to the invention (counting the liquid polyester such as described hereinabove) can range from 0.01% to 30% by weight, in particular from 0.1% to 25% by weight, and more particularly from 0.1% to 20% by weight with respect to the total weight of the composition.

According to a particular embodiment, the composition according to the invention comprises less than 10%, in particular less than 5%, preferably less than 3%, and more preferably less than 2% by water weight with respect to the total weight of said composition.

In particular, the compositions according to the invention can be anhydrous.

Preferably, the compositions according to the invention are anhydrous compositions. The term “anhydrous” particularly infers that water is preferably not deliberately added to the compositions but may be present at trace levels in the various compounds used in the compositions.

In addition to the presence of an oil, the composition according to the invention comprises at least one wax or a mixture of waxes.

Wax(es)

The composition according to the invention comprises at least one wax and can also comprise a mixture of waxes.

The wax or waxes considered in the framework of this invention are in general a lipophilic compound, which is solid at ambient temperature (25° C.), having a reversible solid/liquid change of state and a melting point greater than or equal to 30° C. of up to 200° C. and particularly up to 120° C.

In particular, the waxes suitable for the invention may have a melting point greater than or equal to 45° C., and particularly greater than or equal to 55° C.

According to the invention, the melting point is equivalent to the temperature of the most endothermic peak observed in thermal analysis (DSC) as described in the standard ISO 11357-3; 1999. The melting point of the wax may be measured using a differential scanning calorimeter (DSC), for example the calorimeter sold under the name “DSC 02000” by TA Instruments.

Preferably, the waxes have an enthalpy of fusion ΔHf greater than or equal to 70 J/g.

Preferably, the waxes comprise at least one part suitable for crystallization, visible by means of diffraction X-ray observations.

The measurement protocol is as follows:

A 5 mg sample of wax placed in a crucible is subjected to a first temperature rise from −20° C. to 120° C., at a heating rate of 10° C./minute, and is then cooled from 120° C. to −20° C. at a cooling rate of 10° C./minute and finally subjected to a second temperature rise from −20° C. to 120° C. at a heating rate of 5° C./minute. During the second temperature rise, the following parameters are measured:
the melting point (T_{f}) of the wax, as mentioned above equivalent to the temperature of the most endothermic peak of the fusion curve observed, representing the variation in the difference in power absorbed as a function of the temperature,
ΔHf: the enthalpy of fusion of the wax equivalent to the integral of the overall fusion curve obtained. This enthalpy of fusion of the wax is the quantity of energy required to change the compound from the solid state to the liquid state. It is expressed in J/g.

The wax or waxes can be hydrocarbon, fluorinated and/or silicone waxes and be of plant, mineral, animal, and/or synthetic origin.

The wax(es) can be present with a content greater than or equal to 5% by weight with respect to the total weight of the composition, preferably 7% by weight, preferentially 10% by weight and more preferentially 15% by weight with respect to the total weight of the composition. Preferably, they are present with a content ranging from 12% to 40% by weight, in particular from 14% to 30% by weight, and more preferably from 15% by 25% by weight with respect to the total weight of said composition.

As wax(es), preference can be given to the use of hydrocarbon waxes such as beeswax, lanolin wax, and Chinese insect waxes, rice bran wax, Carnauba wax, Candellila wax, Ouricury wax, Alfa wax, cork fiber wax, sugarcane wax, Japan wax and sumac wax; montan wax, microcrystalline waxes, paraffins and ozokerite waxes; polyethylene waxes, waxes obtained by means of Fisher-Tropsch synthesis and waxy copolymers and the esters thereof.

Mention may also be made of waxes obtained by means of the catalytic hydrogenation of animal or plant oils having C₈-C₃₂ linear or branched fat chains.

Of these, particular mention may be made of isomerized jojoba oil such as the trans isomerized partially hydrogenated jojoba oil manufactured or sold by Desert Whale under the trade name ISO-JOJOBA-50®, hydrogenated sunflower oil, hydrogenated castor oil, hydrogenated coconut oil, hydrogenated lanolin oil, di-(trimethylol-1,1,1 propane) tetrastearate sold under the name “HEST 2T-4S” by HETERENE, di-(trimethylol-1,1,1 propane) tetrabehenate sold under the name HEST 2T-4B by HETERENE.

Silicon waxes can also be mentioned such as alkyl dimethicones or alkoxy dimethicones having 16 to 45 carbon atoms, fluorinated waxes.

The wax can also be used obtained by hydrogenating esterified olive oil with stearyl alcohol sold under the name “PHYTOWAX Olive 18 L 57” or waxes obtained by hydrogenating esterified castor oil with cetyl alcohol sold under the name “PHYTOWAX ricin 16L64 and 22L73”, by SOPHIM. Such waxes are described in the application FR-A-2792190.

The composition can comprise at least one non-polar wax. Preferably, the wax or waxes comprise one or several non-polar waxes chosen from among polyethylene wax, paraffin wax, ozokerite, and mixtures thereof.

The composition can comprise at least one polar wax. The term “polar wax” denotes waxes that comprise in their chemical structure, in addition to carbon and hydrogen atoms, at least one highly electronegative heteroatom, such as O, N or P.

Preferably, the wax or waxes comprise one or several polar and/or non-polar waxes chosen from among carnauba wax, candellila wax, natural (or whitened) beeswax and synthetic beeswax, paraffin and hydrocarbon wax, and mixtures thereof. As synthetic beeswax, mention can be made of the wax sold under the name Cyclochem 326 A by Evonik Goldschmidt (INCI name: Synthetic Beeswax).

Preferably the composition comprises a mixture of polar wax(es) and non-polar wax(es).

The composition can comprise at least one wax that has a hardness ranging from 0.05 MPa to 15 MPa, and preferably ranging from 6 MPa to 15 MPa. The hardness is determined by the measurement of the compression force measured at 20° C. using a texturometer sold under the name TA-TX2i by RHEO, provided with a stainless steel cylinder with a diameter of 2 mm that is displaced at the measuring speed of 0.1 mm/s, and penetrating into the wax at a penetration depth of 0.3 mm.

According to a particular embodiment, the compositions in accordance with the invention can comprise at least one wax referred to as tacky i.e. having a stickiness greater or equal to 0.7 N·s and a hardness less than or equal to 3.5 MPa.

The use of such a tacky wax can in particular allow for the obtaining of a cosmetic composition that is applied easily on the eyelashes, that has a good attaching on the eyelashes and that leads to the formation of a smooth, homogeneous and thickening makeup.

The tacky wax used can in particular have a tackiness ranging from 0.7 N·s to 30 N·s, in particular greater than or equal to 1 N·s, in particular ranging from 1 N·s to 20 N·s, in particular greater than or equal to 2 N·s, in particular ranging from 2 N·s to 10 N·s, and in particular ranging from 2 N·s to 5 N·s.

The tackiness of the wax is determined by the measurement of the change in the force (compression force or stretching force) according to time, at 20° C. using the texturometer sold under the name “TA-TX2i®” by RHEO, provided with a polymer acrylic mobile in the shape of a cone forming a 45° angle.

The measurement protocol is as follows:

The wax is melted at a temperature equal to the melting point of the wax +10° C. The melted wax is poured into a receptacle 25 mm in diameter and 20 mm deep. The wax is recrystallized at ambient temperature (25° C.) for 24 hours in such a way that the surface of the wax is flat and smooth, then the wax is stored for at least 1 hour at 20° C. before carrying out the measurement of the tackiness.

The mobile of the texturometer is displaced at a speed of 0.5 mm/s, then penetrates into the wax to a penetration depth of 2 mm. When the mobile has penetrated into the wax at the depth of 2 mm, the mobile is maintained fixed for 1 second (corresponding to the relaxation time) then is withdrawn at the speed of 0.5 mm/s.

During the relaxation time, the force (compression force) decreases substantially until it becomes zero than, when the mobile is withdrawn, the force (stretching force) becomes negative and then increases again to the value of 0. The tackiness corresponds to the integral of the curve of the force according to time for the portion of the curve that corresponds to the negative values of the force (stretching force). The value of the tackiness is expressed in N·s.

The tacky wax that can be used generally has a hardness less than or equal to 3.5 MPa, in particular ranging from 0.01 MPa to 3.5 MPa, in particular ranging from 0.05 MPa to 3 MPa, and even ranging from 0.1 MPa to 2.5 MPa.

The hardness is measured according to the protocol described hereinabove.

C20-C40 alkyl (hydroxystearyloxy)stearate (the alkyl group comprising 20 to 40 carbon atoms), alone or in a mixture, may be used as the wax, in particular a C₂₀-C₄₀ 12-alkyl (12′-hydroxystearyloxy)stearate having the formula (II):

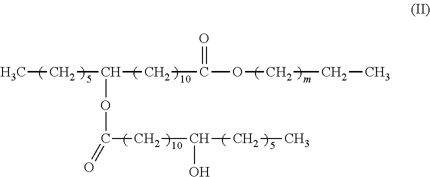

wherein m is an integer ranging from 18 to 38, or a mixture of compounds having the formula (II).

Such a wax is particularly sold under the names “Kester Wax K 82 P®” and “Kester Wax K 80 P®” by KOSTER KEUNEN.

The microcrystalline wax marketed under the reference SP18 by STRAHL can also be used and PITSCH which has a hardness of about 0.46 MPa and a tackiness value of about 1 N·s.

The wax or waxes can be present in the form of an aqueous microdispersion of wax. The term “aqueous microdispersion of wax” refers to an aqueous dispersion of particles of wax, wherein the size of said particles of wax is less than or equal to about 1 μm.

Microdispersions of wax are stable dispersions of colloidal particles of wax, and are in particular described in “Microemulsions Theory and Practice”, L. M. Prince Ed., Academic Press (1977) pages 21-32.

In particular, these microdispersions of wax can be obtained by melting the wax in the presence of a surfactant, and possibly of a portion of the water, then the progressive adding of hot water with stirring. The intermediate formation is observed of an emulsion of the water-in-oil type, followed by a phase inversion with the final is obtaining of a microemulsion of the oil-in-water type. During cooling, a stable microdispersion of solid colloidal particles of wax is obtained.

The microdispersions of wax can also be obtained by stirring the mixture of wax, surfactant and water using means of stirring such as ultrasound, high pressure homogenizer, turbines.

The particles of the microdispersion of wax preferably have average dimensions less than of 1 μm (in particular ranging from 0.02 μm to 0.99 μm), preferably less than 0.5 μm (in particular ranging from 0.06 μm to 0.5 μm).

Preferably, the composition according to the invention comprises a total content in polar wax(es) greater than or equal to 5% by weight with respect to the total weight of the composition, and advantageously in polar hydrocarbon wax. The composition according to the invention advantageously comprises a total content in polar wax(es) ranging from 1% to 30% by weight of wax with respect to the total weight of the composition, preferably from 5% to 20% by weight of wax with respect to the total weight of the composition.

According to one preferred embodiment, the composition according to the invention comprises at least one paraffin wax, and preferably in a content of at least 2% by weight in relation to the total weight of said composition.

According to a particular embodiment, the composition according to the invention comprises a total content in non-polar wax greater than or equal to 0.5% by weight in relation to the total weight of the composition and preferably, from 1% to 15% by weight, in particular from 1.5% to 10% by weight in relation to the total weight of the composition, more preferentially from 1.5% to 9% by weight in relation to the total weight of the composition.

Dyes

The composition according to the invention can comprise at least one dye.

This dye or these dyes are preferably chosen from the group constituted of powder materials, liposoluble dyes, hydrosoluble dyes, and mixtures thereof.

Preferably, the composition according to the invention comprises at least one powder dye. Preferably, the powder dye or dyes can be chosen from among pigments.

Preferably, the pigment or pigments contained in the composition according to the invention are chosen from among metal oxides, preferably iron oxides.

These dyes may be present with a content ranging from 0.01% to 20% by weight with respect to the total weight of the composition, in particular from 1% to 15% by weight, preferably from 3% by 10% by weight with respect to the total weight of the composition.

Preferably, the dye or dyes are chosen from among one or several metal oxides present with a content greater than or equal to 2% by weight with respect to the total weight of the composition, advantageously between inclusively 3% and 12% by weight with respect to the total weight of the composition.

Film-Forming Lipophilic Polymer

The composition according to the invention can comprise at least one lipophilic film-forming polymer.

The lipophilic film-forming polymer may be present in the composition according to the invention in a dry matter content ranging from 0.1 to 20% by weight with respect to the total weight of the composition, preferably from 2% to 15% by weight, and more preferably from 4% to 10% by weight.

In this invention, the term “film-forming polymer” refers to a polymer able to form alone or in the presence of an auxiliary film-forming agent, a macroscopically continuous film that adheres to the keratin fibers, and preferably a cohesive film, and more preferably a film for which the cohesion and mechanical properties are such that said film can be isolated and manipulated in an isolated manner, for example when said film is carried out by pouring on an anti-adherent surface such as a Teflon or silicone surface.

Among the film-forming polymers that can be used in the composition of this invention, mention can be made of radical or polycondensate type synthetic polymers, polymers of natural origin, and mixtures thereof.

Radical Film-Forming Polymers

The term “radical film-forming polymer” refers to a polymer obtained by polymerization of unsaturated monomers in particular ethylenic, with each monomer able to be homopolymerized (contrary to polycondensates).

Radical film-forming polymers can in particular be polymers, or copolymers, vinyl, in particular acrylic polymers.

The vinyl film-forming polymers can result from the polymerization of ethylenic unsaturated monomers that have at least one acid group and/or esters of these acid monomers and/or amides of these acid monomers.

As a monomer carrying an acid group, α,β-ethylenic unsaturated carboxylic is acids can be used such as acrylic acid, methacrylic acid, crotonic acid, maleic acid, itaconic acid. Preferably (meth)acrylic acid and crotonic acid are used, and more preferentially (meth)acrylic acid.

Esters of acid monomers are advantageously chosen from among the esters of (meth)acrylic acid (also called (meth)acrylates), in particular alkyl (meth)acrylates, in particular C1-C30 alkyl, preferably C1-C20 alkyl (meth)acrylates, aryl (meth)acrylates, in particular C6-C10 aryl meth(acrylates), hydroxyalkyl (meth)acrylates, in particular C2-C6 hydroxyalkyl (meth)acrylates.

Alkyl (meth)acrylates include methyl methacrylate, ethyl methacrylate, butyl methacrylate, isobutyl methacrylate, 2-ethyl hexyl methacrylate, lauryl methacrylate, and cyclohexyl methacrylate.

Among the hydroxyalkyl (meth)acrylates, mention can be made of hydroxyethyl acrylate, 2-hydroxypropyle acrylate, hydroxyethyl methacrylate, and 2-hydroxypropyl methacrylate.

Among the aryl (meth)acrylates, mention can be made of benzyl acrylate and phenyl acrylate.

The esters of the (meth)acrylic acid that are particularly preferred are the alkyl (meth)acrylates.

According to this invention, the ester alkyl group can either be fluorinated or be perfluorinated, i.e. a portion or all of the hydrogen atoms of the alkyl group are substituted with fluorine atoms.

As amides of acid monomers, mention can be made for example of (meth)acrylamides, and in particular the N-alkyl (meth)acrylamides, in particular C2-C12 alkyl. Among the N-alkyl (meth)acrylamides, mention can be made of N-ethyl acrylamide, N-t-butyl acrylamide, N-t-octyl acrylamide and N-undecylacrylamide.

The vinyl film-forming polymers can also result from the homopolymerization or from the copolymerization of monomers chosen from among the vinyl esters and the styrene monomers. In particular, these monomers can be polymerized with acid monomers and/or their esters and/or their amides, such as those mentioned hereinabove.

As an example of vinyl esters, mention can be made of vinyl acetate, vinyl neodecanoate, vinyl pivalate, vinyl benzoate and vinyl t-butyl benzoate.

As styrene monomers, mention can be made of styrene and alpha-methyl styrene.

Film-Forming Polycondensates

Among the film-forming polycondensates, mention can be made of polyurethanes, polyesters, polyester amides, polyamides, and epoxyester resins, polyureas.

The polyurethanes can be chosen from among anionic, cationic, nonionic or amphoteric polyurethanes, acrylic polyurethanes, poly-urethanes-polyvinylpyrrolidones, polyester-polyurethanes, polyether-polyurethanes, polyureas, polyurethane polyureas, and mixtures thereof.

The polyesters can be obtained, continuously, by polycondensation of dicarboxylic acids with polyols, in particular diols.

The dicarboxylic acid can be aliphatic, alicyclic or aromatic. The following can be mentioned as examples of such acids: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phtalic acid, dodecanedioic acid, 1,3-cyclohexanedicarboxylic acid, 1,4-cyclohexanedicarboxylic acid, isophtalic acid, terephtalic acid, 2,5-norbornane dicarboxylic acid, diglycolic acid, thiodipropionic acid, 2,5-naphtalenedicarboxylic acid, 2,6-naphtalenedicarboxylic acid. These dicarboxylic acid monomers can be used alone or in combination with at least two dicarboxylic acid monomers. Among these monomers, phtalic acid, isophtalic acid, and terephtalic acid will preferably be chosen.

The diol can be chosen from among the aliphatic, alicyclic, aromatic diols. A diol will be used preferably chosen from among: ethylene glycol, diethylene glycol, triethylene glycol, 1,3-propanediol, cyclohexane dimethanol, 4-butanediol. As other polyols, glycerol, pentaerythritol, sorbitol, trimethylol propane can be used.

The polyester amides can be obtained in a manner similar to the polyesters, by polycondensation of diacids with diamines or amino alcohols. As diamine, ethylenediamine, hexamethylenediamine, meta- or para-phenylenediamine can be used. As aminoalcohol, monoethanolamine can be used.

According to an alternative embodiment of the composition according to the invention, the lipophilic film-forming polymer can be a polymer solubilized in a liquid fatty phase comprising oils such as those described hereinabove (it is then said that the film-forming polymer is a liposoluble polymer). Preferably, the liquid fatty phase comprises a volatile oil, possibly in a mixture with a non-volatile oil, with the oils able to be chosen from among the oils mentioned hereinabove.

As an example of a liposoluble polymer, mention can be made of vinyl ester copolymers (with the vinyl group being directly connected to the oxygen atom of the ester group and the vinyl ester having a linear or branched saturated hydrocarbon radical, with 1 to 19 carbon atoms, linked to the carbonyl of the ester group) and at least one other monomer that can be a vinyl ester (different from the vinyl ester already present), an a-olefin (having from 8 to 28 carbon atoms), an alkylvinylether (of which the alkyl group comprises from 2 to 18 carbon atoms), or an allylic or methallylic ester (having a linear or branched saturated hydrocarbon radical, with 1 to 19 carbon atoms, linked to the carbonyl of the ester group).

These copolymers can be cross-linked using crosslinking agents which can be either of the vinyl type, or of the allylic or methallylic type, such as tetraallyloxyethane, divinylbenzene, divinyl octanedioate, divinyl dodecanedioate, and divinyl octadecanedioate.

As examples of copolymers, the following copolymers can be mentioned: acetate of vinyl/stearate of allyl, acetate of vinyl/laurate of vinyl, acetate of vinyl/stearate of vinyl, acetate of vinyl/octadecylvinylether, propionate of vinyl/laurate of allyl, propionate of vinyl/laurate of vinyl, stearate of vinyl/ethylvinylether, propionate of vinyl/cetyl vinyl ether, stearate of vinyl/acetate of allyl, dimethyl-2, 2 octanoate of vinyl/laurate of vinyl, dimethyl-2, 2 pentanoate of allyl/laurate of vinyl, dimethyl propionate of vinyl/stearate of vinyl, dimethyl propionate of allyl/stearate of vinyl, propionate of vinyl/stearate of vinyl, cross-linked with 0.2% divinyl benzene, dimethyl propionate of vinyl/laurate of vinyl, cross-linked with 0.2% divinyl benzene, acetate of vinyl/octadecyl vinyl ether, cross-linked with 0.2% tetraallyloxyethane, acetate of vinyl/stearate of allyl, cross-linked with 0.2% divinyl benzene, acetate of vinyl/octadecene-1 cross-linked with 0.2% divinyl benzene and propionate of allyl/stearate of allyl cross-linked with 0.2% divinyl benzene.

According to one particular embodiment, the composition according to the invention comprises at least one liposoluble polymer.

In particular, said liposoluble polymer is chosen from among vinyl ester copolymers (with the vinyl group being directly connected to the oxygen atom of the ester group and the vinyl ester having a linear or branched saturated hydrocarbon radical, with 1 to 19 carbon atoms, linked to the carbonyl of the ester group), an allylic or methallylic ester (having a linear or branched saturated hydrocarbon radical, with 1 to 19 carbon atoms, linked to the carbonyl of the ester group).

Preferably, said liposoluble polymer is chosen from the acetate of vinyl/stearate of allyl copolymers.

As liposoluble film-forming polymers, mention can also be made of liposoluble copolymers, and in particular those resulting from the copolymerization of vinyl esters having from 9 to 22 carbon atoms or alkyl acrylates or methacrylates, alkyl radicals having from 10 to 20 carbon atoms.

Such liposoluble copolymers can be chosen from among the copolymers of vinyl polystearate, of vinyl polystearate cross-linked using divinylbenzene, diallylether or diallyl phtalate, copolymers of stearyl poly(meth)acrylate, vinyl polylaurate, lauryl poly(meth)acrylate, with these poly(meth)acrylates able to be cross-linked using ethylene glycol or tetraethylene glycol dimethacrylate.

The liposoluble copolymers defined hereinabove are known and are in particular described in application FR 2 232 303; they can have a mean molecular weight by weight ranging from 2,000 to 500,000 and preferably from 4,000 to 200,000.

According to another embodiment, at least one copolymer resulting from the copolymerization of vinyl esters having from 9 to 22 carbon atoms, and in particular vinyl polylaurate copolymers can be used as a liposoluble copolymer.

According to a particular embodiment, the composition according to the invention comprises at least one liposoluble polymer chosen from among allyl vinyl/stearate acetate copolymers, vinyl polylaurate copolymers and mixtures thereof.

As liposoluble film-forming polymers that can be used in the invention, mention can also be made of polyalkylenes and in particular C2-C20 alkene copolymers, such as polybutene, alkylcelluloses with a linear or branched C1 to C8 alkyl radical, whether or not saturated, such as ethylcellulose and propylcellulose, copolymers of vinylpyrolidone (VP) and in particular copolymers of vinylpyrrolidone and of alkene in C2 to C40 and more particularly in C3 to C20. As an example of VP copolymer that can be used in the invention, mention can be made of vinyl VP/acetate copolymer, ethyl VP/methacrylate, butylated polyvinylpyrolidone (PVP), ethyl/methacrylic acid VP/methacrylate, VP/eicosene, VP/hexadecene, VP/triacontene, VP/styrene, VP/acrylic acid/lauryl methacrylate.

Mention can also be made of silicone resins, generally soluble or swellable in silicone oils, which are cross-linked polyorganosiloxane polymers. The classification of silicone resins is known under the name “MDTQ”, the resin being described according to the various siloxane monomeric units comprised therein, each of the letters “MDTQ” characterizing a type of unit.

As examples of commercially available polymethylsilsesquioxane resins, mention may be made of those marketed:
by Wacker under the reference Resin MK such as Belsil PMS MK
by SHIN-ETSU under references KR-220L.

As siloxysilicate resins, mention can be made of trimethylsiloxysilicate resins (TMS) such as that sold under the reference SR1000 by General Electric or under the reference TMS 803 by Wacker. Mention may further be made of trimethylsiloxysilicate resins marketed in a solvent such as cyclomethicone, sold under the name “KF-7312J” by Shin-Etsu, “DC 749”, “DC 593” by Dow Corning.

Mention can also be made of silicone resin copolymers such as those mentioned hereinabove with polydimethylsiloxanes, such as pressure-sensitive adhesive copolymers sold by Dow Corning under the reference BIO-PSA and described in document U.S. Pat. No. 5,162,410 or silicone copolymers coming from the reaction of a silicone resin, such as those described hereinabove, and of a diorganosiloxane such as described in document WO 2004/073626.

According to an embodiment of the invention, the film-forming polymer is a film-forming linear sequenced ethylenic polymer, that preferably comprises at least one first sequence and at least one second sequence having different vitreous transition temperature (Tg), said first and second sequences being connected together by an intermediate sequence that comprises at least one monomer constituting the first sequence and at least one monomer constituting the second sequence.

Advantageously, the first and second sequences and of the sequenced polymer are incompatible with one another.

Such polymers are described for example in EP 1 411 069 or WO 04/028488.

The lipophilic film-forming polymer can also be present in the composition in the form of particles in dispersion in a non-aqueous phase. As examples of non-aqueous dispersions of film-forming polymer, mention can be made of acrylic is dispersions in isododecane such as Mexomère PAP® from CHIMEX, dispersions of particles of a grafted ethylenic polymer, preferably acrylic, in a liquid fatty phase, with the ethylenic polymer being advantageously dispersed in the absence of additional stabilizer on the surface of the particles such as described in particular in WO 04/055081.

Preferably, the composition according to the invention comprises at least one liposoluble film-forming polymer in particular chosen from among vinyl acetate/alkyl stearate copolymers, vinyl polylaurate copolymers and mixtures thereof.

Lipophilic Gelling Agents

The composition according to the invention can comprise at least one lipophilic gelling agent. This gelling agent can be organic or mineral, polymeric or molecular.

By way of example of a mineral lipophilic gelling agent, mention may be made of optionally modified clays such as hectorites modified by a C₁₀ to C₂₂ fatty acid ammonium chloride, such as hectorite modified with di-stearyl di-methyl ammonium chloride such as, for example, that marketed under the name Bentone 38V® by ELEMENTIS.

Mention may also be made of pyrogenic silica optionally with a hydrophobic surface treatment wherein the particle size is less than 1 μm. Indeed, it is possible to modify the surface of the silica chemically, by means of a chemical reaction giving rise to a reduction in the silanol groups present on the silica surface. The silanol groups may particularly be substituted with hydrophobic groups: a hydrophobic silica is thus obtained. The hydrophobic groups may be:
trimethylsiloxyl groups, particularly obtained by treating pyrogenic silica in the presence of hexamethyldisilazane. Silicas treated in this way are referred to as “Silica silylate” as per the CTFA (6^{th} edition, 1995). They are for example marketed under the references Aerosil R812® by DEGUSSA, CAB-O-SIL TS-530® by CABOT,
dimethylsilyloxyl or polydimethylsiloxane groups, particularly obtained by treating pyrogenic silica in the presence of polydimethylsiloxane or dimethyldichlorosilane. Silicas treated in this way are referred to as “Silica dimethyl silylate” as per the CTFA (6^{th} edition, 1995). They are for example marketed under the references Aerosil R972®, and Aerosil R974® by DEGUSSA, CAB-O-SIL TS-610® and CAB-O-SIL TS-720® by CABOT. The is hydrophobic pyrogenic silica particularly has a particle size that may be nanometric to micrometric, for example ranging from approximately 5 to 200 nm.

The polymeric organic lipophilic gelling agents are, for example, partially or totally cross-linked elastomeric organopolysiloxanes with a three-dimensional structure, such as those sold under the names KSG6®, KSG16® and KSG18® by SHIN-ETSU, Trefil E-505C® and Trefil E-506C® by DOW-CORNING, Gransil SR-CYC®, SR DMF10®, SR-DC556®, SR SCYC Gel®, SR DMF 10 Gel® and SR DC 556 Gel® by GRANT INDUSTRIES, SF 1204® and JK 113® by GENERAL ELECTRIC; ethylcellulose, such as the product sold under the name Ethocel® by DOW CHEMICAL; polyamide-type polycondensates resulting from condensation between (α) at least one acid chosen from among the dicarboxylic acids containing at least 32 carbon atoms such as dimer fatty acids and (β) an alkylene diamine, and in particular ethylene diamine, in which the polyamide polymer comprises at least one terminal carboxylic acid group esterified or amidified with at least one monoalcohol or one monoamine containing from 12 to 30 carbon atoms, and linear and saturated, and in particular ethylenediamine/stearyl dilinoleate copolymers such as the product sold under the name Uniclear 100 VG® by ARIZONA CHEMICAL; galactomannans containing from one to six, and in particular from two to four, hydroxyl groups per monosaccharide, substituted with a saturated or unsaturated alkyl chain, such as guar gum alkylated with C₁ to C₆, and in particular C₁ to C₃ alkyl chains, and mixtures thereof. Block copolymers of the “diblock”, “triblock” or “radial” type, of the polystyrene/polyisoprene or polystyrene/polybutadiene type, such as those marketed under the trade name Luvitol HSB® by BASF, of the polystyrene/copoly(ethylene-propylene) type, such as those marketed under the name Kraton® by SHELL CHEMICAL CO or of the polystyrene/copoly(ethylene-butylene) type, mixtures of triblock and radial (star) copolymers in isododecane, such as those marketed by PENRECO under the trade name Versagel® for instance the mixture of butylene/ethylene/styrene triblock copolymer and of ethylene/propylene/styrene star copolymer in isododecane (Versagel M 5960).

Among the lipophilic gelling agents suitable for use in the compositions according to the invention, mention may also be made of dextrin and fatty acid esters, such as dextrin palmitates, particularly such as those sold under the names Rheopearl TL® or Rheopearl KL® by CHIBA FLOUR.

According to a particular embodiment, the lipophilic gelling agents(s) are chosen from among the mineral lipophilic gelling agents and in particular hectorite is modified with di-stearyl di-methyl ammonium chloride.

According to a particular embodiment, the lipophilic gelling agents(s) are chosen from among the lipophilic polyamide polycondensates.

The term “polycondensate” refers in terms of the invention to a polymer obtained through polycondensation namely by chemical reaction between monomers that have different functional groups chosen in particular from among the acid, alcohol and amine functions.

The term “polymer” in terms of the invention means a compound that has at least 2 repetition patterns, preferably at least 3 repetition patterns and more preferable 10 repetition patterns.

The lipophilic polyamide polycondensates can in particular be chosen from among the polyamide polymers comprising a) polymeric backbone that has hydrocarbon repetition patterns provided with at least one non-pendant amide pattern, and possibly b) at least one pendant fatty chain comprising from 6 to 120 carbon atoms, preferably from 8 to 120 carbon atoms, and more preferably from 12 to 70 carbon atoms, and/or at least one terminal fatty chain that may be functionalized, comprising at least 4 carbon atoms and being linked to these hydrocarbon patterns.

The term “functionalized chains” in terms of the invention refers to an alkyl chain comprising one or several functional group or reagents in particular chosen from among the amides, hydroxyl, ether, oxyalkylene or polyoxyalkylene groups, halogen, of which the fluorinated or perfluorinated groups, ester, siloxane, polysiloxane. Furthermore, the hydrogen atoms of one or several fatty chains can be substituted at least partially with fluorine atoms.

The term “hydrocarbon repetition patterns” in terms of the invention refers to a pattern comprising from 2 to 80 carbon atoms, and preferably from 2 to 60 carbon atoms, carrying hydrogen atoms and possibly oxygen atoms, which can be linear, branched or cyclic, saturated or unsaturated. These patterns further comprise at least one amide group advantageously non-pendant and found in the polymeric backbone.

Advantageously, the pendant chains are directly linked to at least one of the atoms of nitrogen of the polymeric backbone.

The lipophilic polyamide polycondensate can comprise between the hydrocarbon patterns silicone patterns or oxyalkylene patterns in C₂-C₃.

Furthermore, the lipophilic polyamide polycondensate of the composition of is the invention advantageously comprises from 40 to 98% of the fatty chains with respect to the total number of amide patterns and fatty chains and more preferably from 50 to 95%.

Preferably, the pendant fatty chains are linked to at least one of the atoms of nitrogen of the amide patterns of the polymer. In particular, the fatty chains of this polyamide represent from 40 to 98% of the total number of amide patterns and of the fatty chains, and more preferably from 50 to 95%.

Advantageously, the lipophilic polyamide polycondensate has a mean molecular weight by weight less than 100,000 (in particular ranging from 1000 a 100 000), in particular less than 50,000 (in particular ranging from 1000 to 50,000), and more particularly ranging from 1000 to 30,000, preferably from 2000 to 20,000, and more preferably from 2000 to 10,000.

The lipophilic polyamide polycondensate is not soluble in water, in particular at 25° C. In particular, it does not comprise an ionic group.

As preferred lipophilic polyamide polycondensates that can be used in the invention, mention can be made of polyamides branched by pendant fatty chains and/or terminal fatty chains having from 6 to 120 carbon atoms and more particularly from 8 to 120 and in particular from 12 to 68 carbon atoms, with each terminal fatty chain being linked to the polyamide backbone by at least one bond group L. The bond group L can be chosen from among the ester, ether, amine, urea, urethane, thioester, thioether, thiurea, thiourethane groups. Preferably, these polymers comprise a fatty chain at each end of the polyamide backbone.

These polymers are preferably polymers resulting from a polycondensation between a carboxylic diacid having at least 32 carbon atoms (that has in particular from 32 to 44 carbon atoms) with an amine chosen from among diamines that have at least 2 carbon atoms (in particular from 2 to 36 carbon atoms) and triamines having at least 2 carbon atoms (in particular from 2 to 36 carbon atoms). The diacide is preferably a dimer coming from ethylene unsaturation fatty acid having at least 16 carbon atoms, preferably from 16 to 24 carbon atoms, such as oleic, linoleic or linolenic acid. The diamine is preferably diamine ethylene, diamine hexylene, diamine hexamethylene. The triamine is for example triamine ethylene. For polymers comprising one or 2 terminal carboxylic acid groups, it is advantageous to esterify them with a monoalcohol that has at least 4 carbon atoms, preferably from 10 to 36 carbon atoms and more preferably from 12 to 24 and even more preferably from 16 to 24, for example 18 carbon atoms.

The lipophilic polyamide polycondensate of the composition according to the is invention can be in particular chosen from among the polymers having the following formula (A):

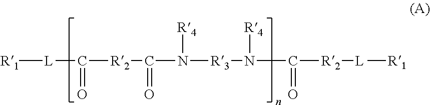

wherein:

n is an integer varying from 1 to 30,

R′₁ represents at each occurrence independently a fatty chain and is chosen from among an alkyl or alcenyl group having at least 4 carbon atoms and in particular from 4 to 24 carbon atoms;

R′₂ represents at each occurrence independently a divalent hydrocarbon chain comprising from 1 to 52 carbon atoms;

R′₃ represents at each occurrence independently a divalent hydrocarbon group, saturated or unsaturated, cyclic or acyclic, possibly substituted and/or possibly interrupted by one or several heteroatoms preferably chosen from among oxygen and nitrogen comprising at least one carbon atoms, in particular R′₃ represents a linear or branched alkylene chain (C₁-C₈); preferably linear alkylene (C₁-C₆) such as ethylene;

R′₄ represents at each occurrence independently: a hydrogen atom, one alkyl group by comprising from 1 to 10 carbon atoms, or a direct link with at least one group chosen from among R′₃ and another R′₄ in such a way that when said group is another R′₄, the nitrogen atom to which are linked both R′₃ and R′₄ is part of a heterocyclic structure defined by R′₄—N—R′₃, with the condition that at least 50% of the R′₄ represent a hydrogen atom, and

L represents a bond group chosen preferably from among an ester or ether or amine or urea or urethane or thioester or thioether or thiurea or thiourethane group, possible substituted by at least one group R′₁ such as defined hereinabove.

According to an embodiment, these polymers are chosen from among the polymers having formula (A) wherein the bond group L represents an ester group ester —C(O)—O— or +O—O(O)—

These polymers are more specially those described in the U.S. Pat. No. 5,783,657 from is Union Camp.

Each one of these polymers satisfies in particular the following formula (B):

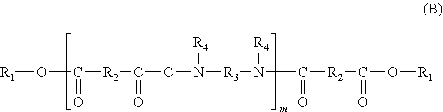

wherein:
m designates an integer of an amid group such that the number of the ester group represents from 10% to 50% of the total number of ester and amide groups;
R₁ represents at each occurrence independently an alkyl or alcenyl group having at least 4 carbon atoms and in particular from 4 to 24 carbon atoms;
R₂ represents at each occurrence independently a C₄ to C₄₂ hydrocarbon group with the condition that 50% of the groups R₂ represent a C₃₀ to C₄₂ hydrocarbon group;
R₃ represents at each occurrence independently a divalent hydrocarbon group, saturated or unsaturated, cyclic or acyclic, possibly substituted and/or possibly interrupted by one or several heteroatoms preferably chosen from among oxygen and nitrogen comprising at least one carbon atoms, in particular R₃ represents a linear or branched alkylene chain (C₁-C₈); preferably linear alkylene (C₁-C₆) such as ethylene; and
R₄ represents at each occurrence independently a hydrogen atom, a C₁ to C₁₀ alkyl group or a direct link to R₃ or to another R₄ in such a way that the nitrogen atom to which are linked both R₃ and R₄ is a part of a heterocyclic structure defined by R₄—N—R₃, with at least 50% of the R₄ representing a hydrogen atom.

In the particular case of the formula (B), the terminal fatty chains possibly functionalized in terms of the invention are terminal chains linked to the last nitrogen atom, of the polyamide backbone.

In particular, the ester groups of the formula (B), which are part of the terminal and/or pendant fatty chains in terms of the invention, represent from 15 to 40% of the total number of ester and amide groups and in particular from 20 to 35%.

In addition, m advantageously represents an integer ranging from 1 to 5 and more particular greater than 2.

Preferably, R₁ is a C₁₂ to C₂₂ alkyl group and preferably C₁₆ to C₂₂. Advantageously, R₂ can be a divalent, saturated or unsaturated, cyclic or acyclic hydrocarbon group in particular R₂ represents a linear or branched (C₁₀-C₄₂) alkylene chain. Preferably, 50% at least and more preferably at least 75% of the radicals R₂ are groups having 30 to 42 carbon atoms. The other R₂ are hydrocarbon groups in C₄ to C₁₉ and even in C₄ to C₁₂.

Preferably, R₃ represents a C₂ to C₃₆ hydrocarbon group or polyoxyalkylene group and R₄ represents a hydrogen atom. Preferably, R₃ represents a C₂ to C₁₂ hydrocarbon group.

The hydrocarbon groups can be linear, cyclic or branched, saturated or unsaturated groups. Moreover, the alkyl and alkylene groups can be linear or branched, saturated or not, groups.

In general, the polymers of the formula (B) have the form of mixtures of polymers, with these mixtures furthermore able to contain a synthesis product that corresponds to a compound of formula (B) where n is equal to 0, i.e. a diester.

According to a particularly preferred form of the invention, a mixture of copolymers of a C36 diacid condensed on diamine ethylene will be used; the terminal ester groups result from the esterification of the terminations of remaining acid by the cetylic, stearylic alcohol or mixtures thereof (also called cetylstearylic) (INCI name: ETHYLENEDIAMINE/STEARYL DIMER DILINOLEATE COPOLYMER). Its mean molar mass by weight is preferably 6000, further preferentially 4000. These mixtures are in particular sold by ARIZONA CHEMICAL under the trade names UNICLEAR 80 and UNICLEAR 100 VG respectively in the form of a at 80% (in active material) in a mineral oil and at 100% (in active material). These mixtures are also sold by CRODA under the trade name OLEOCRAFT LP-10-PA-(MV) respectively at 99.7% (in active material) with a preservative. They have a softening point from 88° C. to 94° C.

In terms of polyamide polycondensates that satisfy the general formula (A), mention can also be made of polymers comprising at least one terminal fatty chain linked to the polymeric backbone by at least one tertiary amid bond group (also called amide terminated polyamide or ATPA). For more information on these polymers, reference can be made to document U.S. Pat. No. 6,503,522.

According to a particularly preferred form of the invention, use will be made more particularly of a copolymer of hydrogenated linoleic diacide, ethylenediamine, di(C14-C18)alkylamine(s) (INCI name: ETHYLENEDIAMIDE/HYDROGENATED DIMER DILINOLEATE COPOLYMER BIS-DI-C14-C18 ALKYL AMIDE). This copolymer is in particular sold under the trade name SYLVACLEAR A200V by ARIZONA CHEMICAL.

According to another embodiment, the polyamide having formula (A) can also be a poly(ester-amide) with ester ends (ester-terminated poly(ester-amide) or ETPEA), as for example those for which the preparation is described in document U.S. Pat. No. 6,552,160.

According to a particularly preferred form of the invention, use will be made more particularly of a copolymer of hydrogenated linoleic diacide, ethylenediamine, neopentylglycol and stearylic alcohol (INCI name: BIS-STEARYL ETHYLENEDIAMINE/NEOPENTYL GLYCOL/STEARYL HYDROGENATED DIMER DILINOLEATE COPOLYMER). This copolymer is in particular sold under the trade name SYLVACLEAR C75 V by ARIZONA CHEMICAL.

As polyamide polycondensates that can be used in the invention, further mention can be made of those that contain at least one terminal fatty chain linked to the polymeric backbone by at least one ether or polyether bond group (it is then referred to as ether terminated poly(ether)amide). Such polymers are described for example in the document U.S. Pat. No. 6,399,713.

The polyamides in accordance with the invention advantageously have a softening temperature greater than 65° C. and are able to range up to 190° C. Preferably, it has a softening temperature ranging from 70 to 130° C. and more preferably from 80 to 105° C. The polyamide is in particular a non-waxy polymer.

As polyamide polycondensates that can be used in the invention, mention can also be made of polyamide resins resulting from the condensing of a aliphatic di-carboxylic acid and of a diamine (including the compounds that have more than 2 carbonyl groups and 2 amine groups), with the carbonyl and amine groups of adjacent unitary patterns being condensed by an amide bond. These polyamide resins are in particular those marketed under the brand Versamid by General Mills, Inc. and Henkel Corp. (Versamid 930, 744 or 1655) or by Olin Mathieson Chemical Corp., under the brand Onamid in particular Onamid S or C. These resins have a mean molar mass by weight ranging from 6000 to 9000. For more information on these polyamides, reference can be made to documents U.S. Pat. No. 3,645,705 and U.S. Pat. No. 3,148,125. More specifically, Versamid 930 or 744 are used.

Polyamides sold by Arizona Chemical under references Uni-Rez (2658, 2931, 2970, 2621, 2613, 2624, 2665, 1554, 2623, 2662) can also be used and the product sold under the reference Macromelt 6212 by Henkel. For more information on these polyamides, reference can be made to document U.S. Pat. No. 5,500,209.

It is also possible to use resins of polyamides coming from vegetables such as those described in U.S. Pat. No. 5,783,657 and U.S. Pat. No. 5,998,570.

Preferably, the lipophilic polyamide polycondensate is a copolymer of a C36 diacid condensed on diamine ethylene; the terminal ester groups result from the esterification of the terminations of remaining acid by the cetylic, stearylic alcohol or mixtures thereof.

Preferably, the composition according to the invention comprises at least one lipophilic gelling agent, in particular at least one mineral lipophilic gelling agent, in particular hectorite modified with di-stearyl di-methyl ammonium chloride and at least one lipophilic polyamide polycondensate and mixtures thereof, in particular a mixture of at least one mineral lipophilic gelling agent and at least one lipophilic polyamide polycondensate.

The lipophilic gelling agent or agents can be present in the composition in a quantity of active material ranging from 0.05% to 12% by weight, preferably from 0.1% to 10% by weight relative to the total weight of the composition.

Fillers

The compositions in accordance with the invention can also comprise at least one filler.

The fillers can be chosen from among those that are well known to those skilled in the art and commonly used in cosmetic compositions. The fillers can be minerals or organic, lamellar or spherical. Mention may be made of talc, mica, silica, kaolin, polyamide powders such as Nylon® marketed under the name Orgasol® by Atochem, poly-β-alanine and polyethylene, tetrafluoroethylene polymer powders such as Teflon®, lauroyl-lysine, starch, boron nitride, polymeric hollow microspheres such as those of polyvinylidene chloride/acrylonitrile such as those sold under the name Expancel® by Nobel Industrie, acrylic powders such as those marketed under the name Polytrap® by Dow Corning, methyl polymethacrylate particles and silicone resin microbeads (Tospearls® from Toshiba, for example), precipitated calcium carbonate, magnesium carbonate and hydro-carbonate, hydroxyapatite, hollow silica microspheres (Silica Beads® from MAPRECOS), glass or ceramic microcapsules, metallic soaps derived from carboxylic organic acids having 8 to 22 carbon atoms, preferably from 12 to 18 carbon atoms, for example zinc, magnesium or lithium stearate, zinc laurate, magnesium myristate.

A compound liable to swell in heat can also be used and in particular thermoexpandable particles such as unexpanded microspheres of copolymer of polyvinylidene/acrylonitrile/methacrylate of methyl chloride or of copolymer of homopolymer of acrylonitrile such as for example those marketed respectively under the references Expancel® 820 DU 40 and Expancel® 007WU by AKZO NOBEL.

The fillers can represent from 0.1% to 10%, in particular from 0.2% to 5%, by weight in relation to the total weight of the composition.

Additives

The compositions in accordance with the invention can also comprise at least one additive.

As an additive that can be used in the compositions in accordance with the invention, mention can in particular be made of pasty fatty bodies, antioxidants, preservatives, perfumes, neutralizers, emollients, thickeners, coalescing agents, plasticizers, moisturizers, vitamins, and mixtures thereof.

Obviously, those skilled in the art will take care to choose these optional additional compounds, and/or the quantity thereof, such that the advantageous properties of the active constituents of the composition according to the invention are not, or are substantially not, altered by the envisaged addition.

Cosmetic Compositions

The present invention also relates to a cosmetic composition including, in a physiologically acceptable medium, a composition such as defined above.

The term “physiologically acceptable medium” is intended to denote a medium that is particularly suitable for the application of a composition of the invention to the skin, the eyelashes or the eyebrows.

The physiologically acceptable medium is generally suitable for the nature of the support to which the composition should be applied, and also for the way in which the composition is to be packaged.

The composition implemented can in particular have the form of a product for eyelashes such as a mascara, or of a product for eyebrows. More preferentially, the invention concerns a mascara. The term “mascara” denotes a composition intended to be applied on the eyelashes. This can be a makeup composition for the eyelashes, a makeup base for eyelashes (also called a base-coat), a composition to be applied on a mascara (also called a top-coat), or a composition for the cosmetic treatment of eyelashes. The mascara is more particularly intended for the eyelashes of human beings, but also for false eyelashes.

Applications

The present invention also relates to a non-therapeutic cosmetic method for coating with keratin materials, in particular the keratin fibers, such as eyelashes, including a step for applying on said keratin materials, in particular the eyelashes, of at least a cosmetic composition as defined hereinabove.

The present invention also relates to a non-therapeutic cosmetic method for makeup with keratin materials, in particular the keratin fibers, such as eyelashes, including a step for applying on the keratin materials, in particular on the eyelashes, of a cosmetic composition as defined hereinabove.

Kit

This invention also has for object a unit, or kit, for packaging and application of a cosmetic composition for coating keratin fibers comprising:
a device for packaging said cosmetic composition for coating keratin fibers such as described hereinabove,
an applicator of said composition.

Said applicator can be integral with a gripping member forming a cover for said packaging device. In other words, said applicator can be mounted in a removable position on said device between a sealed position and a released position of an opening for the distribution of the device for packaging said composition

A kit for coating keratin fibers adapted to the invention can comprise in particular an applicator configured to apply said cosmetic composition for coating keratin fibers, and where applicable a device for packaging adapted to receive said composition.

Applicator

The applicator comprises means making it possible to smooth and/or separate the keratin fibers, such as the eyelashes or the eyebrows, in particular in the form of teeth, hairs, pins or other reliefs.

The applicator is arranged to apply the composition on the eyelashes or the eyebrows, and can comprise for example a brush or a comb.

The applicator can further be used for the finishing of the makeup, on a region of the eyelashes or of the eyebrows with makeup or loaded with the composition.

The brush can comprise a twisted core and hairs taken between the spires of the core, or be carried out yet in another manner.

The comb is for example made of a single piece by molding of plastic material.

In certain embodiments, the element for application is mounted at the end of a rod, which can be flexible, which can contribute to improving the comfort during application.

Packaging Device

The packaging device comprises a receptacle intended to house the composition for coating keratin fibers. This composition can then be taken in the receptacle by immersing the application into the latter.

This applicator can be integral with an element for closing the receptacle. This closing element can form a member for gripping the applicator. This gripping member can form a cover to be mounted removably on said receptacle by any suitable means such as by screwing, snap-fitting, press-fitting or other. Such a receptacle can therefore house in a reversible manner said applicator.

This receptacle may be provided with a squeezing device suited to remove the excess product taken by the applicator.

A method for applying the composition according to the invention on the eyelashes or on the eyebrows can also comprise the following steps:
forming a deposit of the cosmetic composition on the eyelashes or the eyebrows,
leaving the deposit on the eyelashes or on the eyebrows, with the deposit able to dry.

Note that, according to another embodiment, the applicator can form a product receptacle. In such a case a receptacle can for example be provided in the gripping member and an internal channel can connect on the inside this gripping member to the application elements in relief.

Finally, note that the unit for packaging and applying can have the form of a kit, with the applicator and the packaging device able to be housed separately under the same packaging article.

Throughout the application, the term “comprising a” or “including a” means “comprising at least one” or “including at least one”, unless otherwise specified.

Throughout the above description, unless specified otherwise, the term “between x and y” refers to an inclusive range, i.e. the values x and y are included in the range.

### EXAMPLES

A composition according to the invention with a polyester such as defined hereinabove as well as a comparative composition without this polyester were prepared and tested for their volume effect, as well as their properties for application, stability and makeup.

Preparation Protocol for Said Compositions

The compositions were prepared according to a method with the rotor stator.

In a first step, a bentone gel was prepared according to the following protocol: Isododecane was weighed in a beaker then placed in the Rayneri stirrer with dispersing (speed=400 to 600 rpm according to the quantity of solvent) at ambient temperature. Then, the modified hectorite was added by sprinkling into the isododecane, under stirring, still at ambient temperature.

Then, the propylene carbonate was added and the whole was allowed to stand for about 45 minutes at ambient temperature.

Then, the waxy mixture was prepared by melting the waxes in a double-shell skillet by connecting with the oil bath until melting of the waxes (bath setting 120° C.). The whole was placed under Rayneri stirring with the rotor stator as soon as the waxes were melted in order to homogenize.

The pigments were then added to the waxy mixture, still under stirring with the rotor stator, at about 1500-1700 rpm. The oil bath setting was lowered in order to obtain 95° C. in the skillet and the whole was allowed to stand for 20 to 30 minutes.

The bentone gel (prepared as indicated hereinabove) was added, under rotor stator stirring, and the stirring was increased to 2000 rpm. The temperature of the mixture dropped and was left until homogenization of the temperature at 85° C.

The setting of the oil bath was then lowered to 25° C. and the rotor stator stirring was removed and switching to “step by step” blade with the speed reducing adapter (Rayneri setting 400 rpm).

Starting at 40° C., the additives (micro dispersion of waxes, alcohol, active materials, etc.), were added under Rayneri by switching to stirring with dispersing in order to correctly homogenize (Rayneri speed 700 to 1000 rpm). Finally, the whole was again placed under “step by step” blade in order to continue cooling to 25° C.

Cosmetic compositions with and without polyester according to the invention were prepared according to the tables hereinbelow.

Example 1 according to the invention comprises a polyester, while the comparative example 2 does not comprise polyester according to the invention.

| | | Trade name | Example 1 | Example 2 |
|---|---|---|---|---|
| Phase | Ingredients | (supplier) | (Invention) | (comparative) |
| A1 | Rice bran wax | NC 1720 (CERA | 2.80 | 2.80 |
| | | RICA NODA) | | |
| | Vinylpyrrolidone/eicosene copolymer | ANTARON V 220F | 2.00 | 2.00 |
| | | OR GANEX V 220F | | |
| | | (ISP ASHLAND) | | |
| | Carnauba wax | CARNAUBA WAX | 4.70 | 4.70 |
| | | #1 FLAKES N.F. SP | | |
| | | 63 (STRAHL & | | |
| | | PITSCH) | | |
| | Hydrogenated polyester based on fatty acids | VISCOPLAST | 5.00 | — |
| | and butanediol (MW: 1 500)(DILINOLEIC | 14436 H | | |
| | ACID/BUTANEDIOL COPOLYMER) | (BIOSYNTHIS) | | |
| | Paraffin waxes and | SASOLWAX 5603 | 2.80 | 2.80 |
| | stabilized hydrocarbon waxes | (SASOL) | | |
| | Candellila wax | NC 1630 (CERA | 0.10 | 0.10 |
| | | RICA NODA) | | |
| | C18-C38 fatty alcohol stearate hydroxystearoyl | KESTERWAX K82P | 1.00 | 1.00 |
| | (SYNTHETIC BEESWAX) | (KOSTER | | |
| | | KEUNEN) | | |
| | White beeswax | WHITE BEESWAX | 7.30 | 7.30 |
| | | SP 453P (STRAHL | | |
| | | & PITSCH) | | |
| | Vinyl acetate/alkyl acetate copolymer (65/35) | MEXOMERE PQ | 3.30 | 3.30 |
| | | (CHIMEX) | | |
| | Vinyl polylaurate | MEXOMERE PP | 2.20 | 2.20 |
| | | (CHIMEX) | | |
| | Diacide condensate in C36 hydrogenated/ | OLEOCRAFT LP- | 1.00 | 1.00 |
| | diamine ethylene, esterified by stabilized stearylic | 10-PA-(MV) | | |
| | alcohol (ANOX 20) | (CRODA) | | |
| | (ETHYLENEDIAMINE/STEARYL DIMER | | | |
| | DILINOLEATE COPOLYMER) | | | |
| A2 | Talc | LUZENAC | 1.00 | 1.00 |
| | | PHARMA M | | |
| | | (IMERYS) | | |
| B | Isododecane | ISODODECANE | qsp 100 | qsp 100 |
| | | (INEOS) | | |
| | Black iron oxide | SUNPURO BLACK | 4.20 | 4.20 |
| | | IRON OXIDE C33- | | |
| | | 7001 (SUN) | | |
| C | Modified distearyl dimethyl ammonium hectorite | BENTONE 38 VCG | 5.80 | 5.80 |
| | | (ELEMENTIS) | | |
| D | Propylene carbonate | ARCONATE | 1.90 | 1.90 |
| | | PROPYLENE | | |
| | | CARBONATE | | |
| | | (LYONDELL) | | |
| E | Microdispersion of carnauba wax in water | MEXORYL SAP | 7.00 | 7.00 |
| | | (CHIMEX) | | |
| F | preservative | | qs | qs |

Makeup Tests

The compositions 1 (invention) and 2 (comparative) were then tested for their makeup properties.

In particular, it was observed that the composition according to the invention is significantly more charging and that the progression of the deposit is better compared to the comparative composition without polyester.

As such, the adding of the polyester according to the invention makes it possible to improve the volume effect on the eyelashes compared to a composition without polyester.

Moreover, additional tests were carried out with regards to the stability to friction, stability to water and stability to squalene, as well as the removal of the is mascara.

It was observed that the composition according to the invention has characteristics similar to a conventional waterproof mascara. In particular, the composition of the invention has stability properties that are identical to those of a conventional waterproof mascara and, likewise, the composition of the invention has makeup removal properties that are identical to those of a conventional waterproof mascara.

1. A composition comprising:
at least one oil,
at least one wax, and
at least one liquid polyester obtained by condensing unsaturated fatty acid dimers and/or trimers and diol, according to a content strictly less than 12% by weight with respect to the total weight of said composition.

2. The composition according to claim 1, wherein the fatty acids are chosen from among mono or polyunsaturated fatty acids comprising from 14 to 22 carbon atoms.

3. The composition according to claim 1, wherein the polyester is obtained by condensing an unsaturated fatty acid dimer comprising 36 carbon atoms and 2 carboxylic acid functions.

4. Composition according to claim 1, wherein the diol is a saturated linear diol, preferably a butanediol.

5. The composition according to claim 1, wherein the polyester is a polyester of dilinoleic acid and of 1,4-butanediol.

6. The composition according to claim 1, wherein the content by weight of polyester is less than or equal to 10% with respect to the total weight of said composition.

7. The composition according to claim 1, wherein the wax or waxes comprise(s) one or several polar and/or non-polar wax(es).

8. The composition according to claim 1, comprising a content in wax(es) ranging from 12% to 40% by weight with respect to the total weight of the composition.

9. The composition according to claim 1 comprising at least one volatile oil and/or at least one additional non-volatile oil, and mixtures thereof.

10. The composition according to claim 9, comprising a total content in volatile oil(s) ranging from 0.1% to 80% by weight with respect to the total weight of the composition.

11. The composition according to claim 1, comprising less than 10% of water by weight with respect to the total weight of said composition.

12. The composition according to claim 1, comprising at least one lipophilic film-forming polymer.

13. The composition according to claim 1, further comprising at least one lipophilic gelling agent and at least one lipophilic polyamide polycondensate and mixtures thereof.

14. A method for coating keratin fibers comprising a step of applying on said keratin fibers of a composition according to claim 1.

15. The method Use according to claim 14 wherein said keratin fibers are eyelashes and wherein said method gives rise to a volume effect on the eyelashes.

16. The composition according to claim 2, wherein the polyester is obtained by condensing an unsaturated fatty acid dimer comprising 36 carbon atoms and 2 carboxylic acid functions.

17. The composition according to claim 1, wherein the content by weight of polyester is less than or equal to 7% with respect to the total weight of said composition.

18. The composition according to claim 1, wherein the content by weight of polyester is less than or equal to 5% with respect to the total weight of said composition.

19. The composition according to claim 2, wherein the content by weight of polyester is less than or equal to 10% with respect to the total weight of said composition.

20. The composition according to claim 3, wherein the content by weight of polyester is less than or equal to 10% with respect to the total weight of said composition.
